(19) United States
(12) Patent Application Publication
(10) Pub. No.: US 2008/0085247 A1
(43) Pub. Date: Apr. 10, 2008

(54) Composition of Menthyl Lactate and a Mixture of Menthol Isomers

(75) Inventors: Roland Langner, Bevern (DE); Reinhard Nowak, Mt. Pleasant, SC (US)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: SYMRISE GMBH & CO. KG, Holzminden (DE)

(21) Appl. No.: 11/576,917

(22) PCT Filed: Oct. 13, 2004
(86) PCT No.: PCT/EP04/52529

§ 371 Date: Apr. 9, 2007

(51) Int. Cl. A61K 8/37; A23G 4/06; A23L 1/22; A23L 2/56; A61Q 1/12; A61Q 15/00; A61Q 19/10; A61Q 5/02; C11B 9/00; C12G 3/06; C11D 3/20; A61Q 9/02; A61Q 5/00; A61Q 17/04; A61Q 11/00; A24B 15/30; A61K 31/22; A61K 8/33

(52) U.S. Cl. 424/49; 131/274; 424/59; 424/65; 424/69; 424/73; 426/3; 426/534; 426/590; 426/592; 510/119; 510/130; 510/137; 510/276; 510/488; 510/515; 512/26; 514/546

(57) ABSTRACT

Described is a composition (i) comprising or (ii) essentially consisting of or (iii) consisting of (a) methyl lactate, (b) neomenthol, and (c) menthol, and optionally (d) neoisomenthol and/or (e) isomenthol, wherein said composition has a solidification point below +5° C.

### BACKGROUND OF THE INVENTION

1. Field of the Invention

The present invention relates to the application of a mixture of menthyl lactate, neomenthol and menthol as cooling agent or flavoring agent. In particular, this invention relates to a coolant composition comprising menthyl lactate, neomenthol and menthol having a solidification point below +5° C.

2. Description of the Prior Art

Menthol is a physiological cooling agent well known to the person skilled in the art for its analgesic, freshening and flavoring effects on the skin and/or mucous membranes.

Being a major constituent of peppermint oil, menthol has been used extensively in foodstuff, beverages, dentifrices, mouthwashes, skin and hair care preparations, confectionary, tobacco and pharmaceutical products.

A strong initial cooling sensation can be achieved if products containing menthol are applied to the skin and/or mucous membrane. However, very often there is a need for products containing a coolant composition in order to provide for a long-lasting cooling sensation.

Menthyl lactate also is an important ingredient that is valued for its cooling properties. Compared to menthol, one of the advantages of menthyl lactate is its long-lasting cooling sensation as well as low volatility. However, menthyl lactate does not produce an initial cooling sensation as strong as menthol.

The disadvantage of an addition of menthyl lactate to products is that it has to be either melted at a temperature of 50-60° C. or dissolved in perfume oils, cosmetic oils, or glycol solvents such as 1,2-propylene glycol or dipropylene glycol. When adding menthyl lactate to shampoos, shower gels or foam bath products, it is advisable to first dissolve it in perfume oil or glycol solvents. The resulting solution can then be mixed with the surfactants required.

Menthyl lactate is available from Symrise GmbH & Co. KG (Germany) in two forms, the crystalline and the non-crystalline form:

The non-crystalline form (solidified distillate) has a congealing point of min. 40° C. and an appearance of solid, white substance, while the crystalline form has a congealing point of min. 42° C. and an appearance of white, crystalline powder (EP 0 794 169). The non-crystalline form has the disadvantage of less convenient handling because the product has to be melted in order to remove it from the container, which means that the container has to be heated together with the product.

The crystalline form has the advantage of easy handling. However, it is not always convenient or easy to mix the powder into foodstuff, oral care or cosmetic product bases, which may be in a liquid or a paste-like form. To solve this problem, the crystals can be solubilized using an acceptable solvent, before adding the solution to such a product base. However, this solubilization constitutes an extra processing step for the formulator, making it inconvenient and more costly to use.

There have been reports in the literature where menthol and menthyl lactate have been used in the same formulation by adding menthol and menthyl lactate separately. For example, WO 00/42983 describes a cosmetic composition containing menthol and menthyl lactate, having little odor and being non-irritating. The composition comprises 0.01% to 2% by weight menthol and 0.1% to 10% by weight menthyl lactate, the menthol/menthyl lactate ratio lying in the range of 1/1 to 1/10, preferably 1/6 to 1/2, and being such that the odor of the menthol is barely perceivable, said composition not being irritating, in particular for the sensitive parts of the human body, while conserving the various beneficial effects of menthol.

JP 06329528 and JP10231238 relate to cosmetic composition where menthol and menthyl lactate were used in the same formulation by adding them separately in a ratio of 1/1 and 2/1, respectively. In JP 06329528, the menthol and menthyl lactate concentration ranged from 0.1%:0.1% (ratio 1:1) to 0.2%:0.2% (ratio 1:1) in the examples, while it was claimed that the menthol concentration can be in the range of 0.001-10.0% (w/w) and menthyl lactate can also be added in the range of 0.001-10.0%. In JP 10231238, it was reported that 0.2% menthol and 0.1% menthyl lactate be used in the same formulation with a ratio of 2:1.

Attempts to provide liquid mixtures of menthol and menthyl lactate mixtures that solve the above mentioned problems have been published.

WO 2004/037764 refers to a solution consisting essentially of menthyl lactate and menthol carboxamide dissolved in a solvent, wherein the final menthyl lactate concentration is higher than that achievable by dissolving menthyl lactate in a neat solvent, by a) liquefying menthyl lactate, and b) combining the liquefied menthyl lactate with menthol carboxamide and the solvent, the solvent being acceptable for food, oral care or cosmetic use.

US 2004/0018954 discloses mixtures containing menthol and menthyl lactate, characterized in that it comprises menthol and menthyl lactate in a ratio by weight in the range of 1:4 to 4:1 and the corresponding crystallization point is below room temperature of 25° C. Such a composition which is liquid at room temperature was produced by stirring menthol and menthyl lactate in solid form within the mentioned ratio without heating, i.e. without melting the components. The lowest crystallization point reported was 8.2° C. for a mixture of 50% l-menthol and 50% l-menthyl l-lactate.

Although US 2004/0018954 states that the term “menthol” and “menthyl lactate” covers not only all such stereoisomers, but also any racemate or mixture of said stereoisomers, the authors only investigated and used the common and widely available commercial products l-menthol and l-menthyl l-lactate.

One main disadvantage of the liquid compositions proposed in the prior art is that the solidification points achievable are not low enough to enable storage at temperatures below +5° C. or lower without solidifying, especially not for a prolonged period of time. From an industrial point of view this disadvantage is significant since a product, once it has been solidified within a container (typically barrel or tank), needs to be melted before removal therefrom, which means that the container has to be heated together with the product with all the disadvantages outlined above.

### OBJECTS OF THE INVENTION

The main object of the present invention is to provide a composition containing menthyl lactate and menthol that maintains its liquid state at temperatures below +5° C., preferably without the need of adding a conventional solvent or solubilizer, which can be added directly in liquid form into a formulation of a product without the need of heating or melting of said coolant composition prior to addition to said formulation, being non-irritating and of little odor.

Another object of the present invention is to provide a coolant composition that combines both a strong initial cooling sensation and a long-lasting cooling sensation to a product when applied on the skin and/or mucous membrane.

Accordingly, there remains a need to provide menthyl lactate in liquid form, which is easy to use in further formulations or process operations and which remains stable in liquid form at temperatures below +5° C., preferably at 0° C. or below even during a prolonged period of storage.

### SUMMARY OF THE INVENTION

Surprisingly, it has now been found that a (coolant) composition comprising menthyl lactate and having a solidification point of below +5° C. can be obtained when menthyl lactate is combined, mixed or co-dissolved with a favourably (liquid) mixture of neomenthol, menthol and optionally neoisomenthol and/or isomenthol.

In a first aspect the present invention refers to a (coolant) composition comprising menthyl lactate, neomenthol and menthol and optionally neoisomenthol and/or isomenthol having a solidification point below +5° C. The composition of the invention does not require the addition of a conventional solvent. Typically, the coolant compositions according to the present invention are dear liquid solutions at temperatures between +5° C. and −20° C.

Within the concept of the present invention a method is provided for preparing a clear liquid solution containing menthyl lactate and menthol, wherein the final menthyl lactate concentration is higher than that achievable by mixing menthyl lactate and menthol alone, by combining menthyl lactate and [a (liquid) mixture] of neomenthol and menthol.

It was also found that a composition according to the present invention provides both a strong initial cooling sensation and a long-lasting cooling sensation. It has also been found that the cooling sensation of the (coolant) composition comprising menthyl lactate, neomenthol and menthol taught herein are often not merely additive, but a synergistic cooling sensation often were observed.

Thus, the use of the (coolant) compositions comprising menthyl lactate, neomenthol and menthol as a cooling and/or flavoring agent is a further aspect of the present invention.

A (coolant) composition of the present invention may be employed in a wide variety of products for consumption by or application to the human body, e. g., foodstuff or oral care products such as toothpastes and mouthwashes, chewing gums, confectionery, alcoholic and non-alcoholic beverages, cosmetic and toiletry products such as shaving products, after-shave products, hair and skincare products, bath and shower products, face cleansing products, deodorants and antiperspirants, sun protection and sun tan products, after sun products, anticellulite/slimming products, make-up products e.g. lip sticks, talcum powders, fragrances, and other products such as detergents, fabric softeners, soaps, tobacco products, medicaments etc.

A further aspect of the invention is a product, preferably foodstuff, oral care or cosmetic product into which has been added an amount of a (coolant) composition according to the present invention sufficient to provide a cooling effect. Such foodstuff, oral care or cosmetic product may be provided with the (coolant) composition according to the present invention by any useful method, e. g., by simple addition to, and mixing into the (foodstuff, oral care or cosmetic) product bases an effective amount of the coolant composition according to the present invention.

A method of preparing a product according to the present invention particularly relevant in practice comprises the following steps:
storing a composition according to the present invention at a temperature (i) below +5 ° C. (e. g. in an outside container during wintertime) but (ii) above the solidification point of said composition,
mixing the composition with components of the product base to form the product,
   wherein said composition is not solidified between storing and mixing.

### DETAILED DESCRIPTION OF THE INVENTION

Unexpectedly, the (coolant) compositions according to the present invention comprising menthyl lactate, neomenthol, menthol and optionally neoisomenthol and/or isomenthol have significantly lower solidification points than those reported in US 2004/0018954 although neomenthol and neoisomenthol and/or isomenthol have higher melting points than the corresponding menthol-isomer (vide infra).

Further it was found that (coolant) compositions according to the present invention comprising menthyl lactate, neomenthol, menthol and optionally neoisomenthol and/or isomenthol allow more variations regarding the weight ratios of menthyl lactate and menthol-isomers than indicated in US 2004/0018954.

It was further observed that certain mixtures according to the present invention of menthyl lactate, neomenthol, menthol and optionally neoisomenthol and/or isomenthol yield a shelf-stable liquid product with a solidification point below −20° C. A mixture of neomenthol, menthol and optionally neoisomenthol and/or isomenthol allows dissolution of menthyl lactate in varying amounts, the resulting composition having a low solidification point as described in more detail below.

The general formula for menthyl lactate is shown as follows:

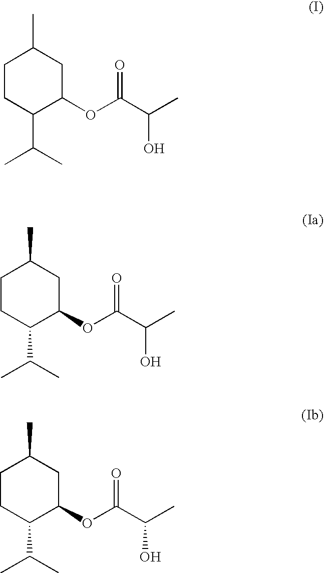

Menthyl lactate may be obtained by reacting menthol with lactic acid to form the corresponding ester. Menthyl lactate contains 4 asymmetrical carbon atoms. It therefore exists in 16 different stereoisomers, whereas the commonly used ones (formula Ia) are made from l-menthol and l-lactic acid or d-lactic acid. L-menthyl l-lactate (formula (Ib)) has a congealing point of 40-42° C. and is commercially available.

Menthol can be obtained naturally from essential oils, e.g. peppermint oil, or can be of synthetic origin. Menthol contains three asymmetrical carbon atoms. Therefore exist 8 different stereoisomers comprising menthol, neomenthol, isomenthol, and neoisomenthol. However, only the l-menthol (i. e. (−)-menthol) of formula (IIa) has been widely used, and its melting point is 42-43° C.

Menthol and its isomers can be illustrated by the following formulas, showing one enantiomer each of the four diastereomers.

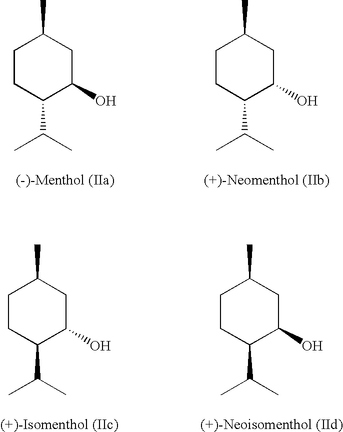

The enantiomers (IIa) to (IId) and their optical antipodes may, for example, be obtained by hydrogenation of thymol (e.g. WO 2004/018398 and the references cited therein) or via cyclization of citronellal to the corresponding isopulegol-isomers and subsequent hydrogenation. The isomers can be separated via accurate distillation (for more details on manufacturing and separation of menthol isomers see “Common Fragrance and Flavor Materials”, 4th Edition, Wiley-VCH, Weinheim 2001, 52-55).

Racemic neomenthol (i.e. a 1:1-mixture of formula (IIb) and its optical antipode) has a melting point of about 53° C. It should be mentioned that neomenthol, regardless its stereoisomeric form, does not exhibit a strong cooling sensation when compared to menthol, in particular when compared to l-menthol. Racemic and enantiopure neomenthol is commercially available, e.g. from Sigma-Aldrich.

The present invention provides a (coolant) composition with a solidification point below +5° C., the composition comprising a) menthyl lactate, b) neomenthol and c) menthol, wherein preferably the weight ratio of a) to the sum of b) and c) is in the range of 6:1 to 1:15 and the weight ratio of b) to c) is in the range 20:1 to 1:10.

In a preferred embodiment the weight ratio of a) to the sum of b) and c) is in the range of 4:1 to 1:10, more preferred in the range of 2:1 to 1:10 and particularly preferred in the range of 1:2 to 1:8.

In a preferred embodiment the weight ratio the weight ratio of b) to c) is in the range of 15:1 to 1:3, more preferred in the range of 10:1 to 1:1 and particularly preferred in the range of 6:1 to 2:1.

Particularly preferred coolant compositions according to the present invention comprise menthyl lactate, neomenthol and menthol in a total amount of 80 to 100%, preferably 85 to 100%, by weight, based on the total weight of the coolant composition according to the present invention.

A (coolant) composition according to the present invention has a solidification point below +5° C., preferably a solidification point of 0° C. or below, more preferably of −10° C. or below.

Preferably in the (coolant) compositions according to the present invention racemic menthol and/or racemic neomenthol are used.

In a particularly preferred embodiment a (coolant) composition according to the present invention comprises neoisomenthol (formula (IId) and/or its enantiomer), preferably racemic neoisomenthol, in an amount of up to 10%, preferably 3 to 8%, by weight, based on the total weight of the coolant composition according to the present invention. Racemic neoisomenthol has a melting point of about 52-53° C.

The following Table 1 indicates solidification points of different mixtures. At 25-30° C. mixtures were prepared by combining and homogenizing the amounts indicated by weight of l-menthyl l-lactate and racemic neomenthol and/or racemic menthol. The resulting mixtures were then stored for 48 hours at temperatures of +4° C., −10° C. or −20° C. respectively and their solid or liquid state determined visually.

TABLE 1

| | Menthyl- | | | | | |
|---|---|---|---|---|---|---|
| | lactate | Neomenthol | Menthol | | | |
| Trial | % wt | % wt | % wt | +4° C. | −10° C. | −20° C. |
| A | 80 | — | 20 | solid | | |
| B | 85 | 15 | — | solid | | |
| C | 70 | 30 | — | liquid | solid | solid |
| D | 60 | 40 | — | liquid | liquid | solid |
| E | 40 | 60 | — | solid | | |
| F | 70 | 20 | 10 | liquid | liquid | liquid |
| G | 60 | 30 | 10 | liquid | liquid | liquid |
| H | 50 | 28 | 22 | liquid | liquid | liquid |
| I | 40 | 40 | 20 | liquid | liquid | liquid |
| J | 30 | 50 | 20 | liquid | liquid | liquid |
| K | 20 | 50 | 30 | liquid | liquid | liquid |
| L | 15 | 65 | 20 | liquid | liquid | liquid |

In a particularly preferred embodiment a coolant composition according to the present invention comprises or essentially consists (i.e. the sum of menthyl lactate, neomenthol, menthol, neoisomenthol, and isomenthol used is equal to or greater than 96%, preferably equal to or greater than 98%, by weight) of
a) 10-40%, preferably 10-30%, more preferably 10-20%, of menthyl lactate,
b) 25-75%, preferably 40-75%, more preferably 50-70%, of neomenthol,
c) 5-30%, preferably 10-30%, more preferably 10-25%, of menthol,
d) 0-10%, preferably 3-8%, more preferably 4-7%, of neoisomenthol, and
e) 0-10%, preferably 0.5-3%, more preferably 0.5-2%, of isomenthol,
   by weight, based on the total weight of the coolant composition according to the present invention.

According to a further particularly preferred embodiment a coolant composition according to the present invention further comprises 0.1-3%, more preferably 0.5-2%, by weight of menthone and/or isomenthone.

Solidification points below −30° C., were obtained with coolant compositions comprising l-menthyl l-lactate, racemic neomenthol, racemic menthol and, optionally, racemic neoisomenthol and/or racemic isomenthol.

The coolant composition of the invention exhibits excellent storage stability. It has been observed that neither menthyl lactate nor neomenthol nor menthol nor the optional components neoisomenthol and isomenthol precipitate from the liquid, even at very low storage temperatures of about −20° C. and even at temperatures of about −30° C. Thus, a coolant composition comprising menthyl lactate, neomenthol, menthol and optionally neosiomenthol or isomenthol is provided without having the aforementioned disadvantages of a solid product.

Furthermore, due to its liquid form, it is considerably easier to manufacture products containing the (coolant) compositions according to the present invention, particularly foodstuff, oral care and cosmetic products, rather than using a powder or an other solid mass which has to be melted prior to use.

The sensory properties of the different menthol-isomers are described in Rivista Italiana EPPOS 1995 Special edition, 31.08.-Feb. 9, 1995, 111-115. It is well known that menthol, most particularly l-menthol, exhibits the strongest and freshest sensory profile among menthol and its isomers.

The presence of menthol (and the isomers neomenthol and/or neoisomenthol and/or isomenthol to a smaller extent) in the coolant composition according to the present invention provides a strong initial cooling sensation, and the menthyl lactate portion provides the long-lasting cooling sensation. Furthermore, surprisingly it has been found that the cooling sensation of the coolant composition according to the present invention comprising menthyl lactate, neomenthol, menthol and optionally neoisomenthol and/or isomenthol often not merely reflects the additive effects of the ingredients. Unexpectedly, when coolant compositions of the present invention were tested by trained panelists in sensory evaluations against menthyl lactate, neomenthol or menthol individually, a synergistic cooling sensation was often observed. This surprising synergistic effect is described in more detail in the examples presented below.

A coolant composition according to the present invention, as a cooling and/or flavoring agent, has the advantage of providing both a strong initial cooling sensation as well as a long-lasting cooling sensation.

Coolant compositions according to the present invention are therefore eminently suitable for incorporation into foodstuff, oral care and cosmetic products in order to deliver the desired cooling sensation by consumption or application to the human body.

The presence of (racemic) neomenthol in a coolant composition according to the present invention reduces or even eliminates the harshness caused by menthol, in particular when using l-menthol, without reducing the overall cooling sensation when compared to the use of l-menthol alone.

In view of the teaching of the present invention the skilled formulator in the art is able to adjust an appropriate ratio of menthyl lactate to neomenthol, menthol and optionally neoisomenthol and/or isomenthol in order to achieve any desired sensory effect as a matter of routine or by a few routine experiments.

A coolant composition of the present invention may be prepared according to the following procedure: under stirring solid menthyl lactate is either directly dissolved in a liquid mixture comprising or consisting of neomenthol, menthol and optionally neoisomenthol and/or isomenthol or alternatively first liquefied or melted, typically at a temperature of about 40° C. to 50° C., prior to addition to the liquid mixture of neomenthol, menthol and optionally neoisomenthol and/or isomenthol. The procedure results in a shelf-stable coolant composition of the present invention.

Alternatively, the coolant composition of the present invention may be formed by liquefying or melting menthyl lactate, typically at a temperature of about 40° C. to 50° C., and adding neomenthol, menthol and optionally neoisomenthol and/or isomenthol, preferably in form of a (liquid) mixture, under stirring. The procedure results in a shelf-stable coolant composition of the present invention.

As stated hereinabove, the (coolant) compositions of the present invention are provided in a liquid form even below +5° C. that exhibits excellent shelf life with no precipitation. This major advantage allows storage of the coolant composition under conditions of varied humidity and temperature e. g. in the range of −20° C. to 40° C., makes it easy to handle and to add directly into formulations without heating, thereby saving time, energy and money. Additionally, the liquid coolant composition of the present invention can be used for manufacturing products without heating. Examples of other cooling substances which advantageously can be combined with a coolant composition according to the present invention and incorporated into products, such as products for direct consumption by or application to the human body, are, for example, substances having a physiologically cooling action, that is to say substances which cause an impression of cold on human skin and/or in the mucous membranes. Suitable substances having a cooling action are, for example, 1-isopulegol, menthone glycerol acetal, substituted menthane-3-carboxamides (for example N-ethylmenthane-3-carboxamide), 2-isopropyl-N,2,3-trimethylbutanamide, substituted cyclohexanecarboxamides, 3-menthoxy-1,2-propanediol, 2-hydroxyethylmenthyl carbonate, 2-hydroxypropylmenthyl carbonate, N-acetylglycine menthyl ester, menthylhydroxycarboxylic esters (for example menthyl 3-hydroxybutyrate), menthyl monosuccinate, 2-mercaptocyclodecanone, and menthyl 2-pyrrolidin-5-one carboxylate, l-menthyl methyl ether.

For use in food and oral care products, a coolant composition of the present invention are favorably employed at levels of 0.01% to 5% by weight, preferably from 0.05% to 3%, more preferably from 0.1% to 2% by weight, based on the total weight of the food or oral care product.

For use in cosmetic products, the coolant compositions of the present invention are favorably employed at levels of 0.01% to 10% by weight, preferably from 0.05% to 5%, more preferably from 0.1% to 2% by weight, based on the total weight of the cosmetic product. However, it is understood that the skilled person may employ the inventive coolant compositions in amounts outside the aforementioned ranges to achieve sensorial effects as desired.

Products in which the coolant composition of the present invention advantageously may be incorporated are many and varied. These products include a wide variety of products for consumption by or application to the human body. The following illustrate the range of products into which coolant compositions of the present invention can be incorporated: edible or potable products including alcoholic and non-alcoholic beverages, confectionery, chewing gum, ice cream, jellies. cosmetics and toiletries including after shave lotions, shaving soaps, creams and foams; toilet water, deodorants and antiperspirants, “solid colognes”, toilet soaps, bath foams, oils and salts; shower gels, body wash, hair shampoos, oils, conditioners, tonics, creams, gels and antidandruff shampoos; talcum powders, face creams, lotions, tonics and gels; hand creams, hand and body lotions, anticellulite/slimming creams and lotions, sunscreen lotions, balms, gels, sprays and creams; after sun lotions, sprays and gels; lip sticks, cleansing tissues, dentifrices, toothpicks, mouthwashes. Other products are pharmaceutical products such as antiseptic ointments, liniments and lotions, cough mixtures, oral analgesic, tobacco preparations, detergents, fabric softeners, water soluble adhesive products for envelopes, postage stamps, adhesive labels etc.

The coolant compositions according to the present invention are essentially insoluble in water. It is therefore preferred to incorporate them into products in a way so that they remain stably distributed throughout the products. This can be done by forming a dispersion, emulsion, or microemulsion, with the compositions solubilized in a suitable solvent that then becomes part of the dispersion or emulsion (including microemulsions). Emulsion systems include oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. Suitable solvents for the compositions according to the present invention include lipophilic or nonpolar solvents such as diethylene glycol, dipropylene glycol, C₁-C₆ alcohols, acetone, and other organic solvents, many of which are also suitable for use as perfume solvents.

Products comprising coolant compositions according to the present invention can, depending on their formulation, be used, for example, as creams, gels, lotions, alcoholic and aqueous/alcoholic solutions, emulsions or stick preparations. These products may also comprise, as further auxiliaries and additives, mild surfactants, co-emulsifiers, superfafting agents, pearlescent waxes, bodying agents, thickeners, polymers, silicone compounds, fats, waxes, stabilizers, biogenic active ingredients, deodorant active ingredients, antidandruff agents, film formers, swelling agents, hydrotropic agents, preservatives, insect repellants, tanning agents, artificial self-tanning agents (e.g. dihydroxyacetone), stabilizers, antioxidants, perfume oils, dyes, antimicrobial agents and the like. The amounts of cosmetic or dermatological auxiliaries and carrier and perfume which can be used in each case can be determined easily by the person skilled in the art by simple trial and error, depending on the nature of the product in question.

A lipid phase of products comprising a coolant composition of the present invention can advantageously be chosen from the following group of substances: mineral oils, mineral waxes; oils, such as triglycerides of capric or of caprylic acid, and also natural oils, such as, for example, castor oil; fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids; alkyl benzoate; silicone oils, such as dimethylpolysiloxane, diethylpolysiloxane, diphenylpolysiloxane, and mixed forms thereof.

The aqueous phase of products comprising a coolant composition according to the present invention advantageously comprises one or more of the following substances: alcohols, diols or polyols (lower alkyl), and ethers thereof, preferably ethanol, isopropanol, propylene glycol, glycerol, ethylene glycol-monoethyl or monobutyl ether, propylene glycol monomethyl, -monoethyl or monobutyl ether, diethylene glycol monomethyl or -monoethyl ether and analogous products, and also alcohols (lower alkyl), e.g. ethanol, 1,2-propanediol, glycerol, and, in particular, one or more thickeners which can advantageously be chosen from the group of silicon dioxide, aluminum silicates, polysaccharides and derivatives thereof, e.g. hyaluronic acid, xanthan gum, hydroxypropylmethylcellulose, particularly advantageously from the group of polyacrylates, preferably a polyacrylate from the group of so-called Carbopols, for example, Carbopol grades 980, 981, 1382, 2984, 5984, in each case individually or in combination.

Cosmetic and/or dermatological products comprising a coolant composition according to the present invention can have the customary constitution and can be used to convey a refreshing/cooling sensation to the human body, and also for the treatment, care and cleansing of the skin and/or of the hair and as a make-up product in decorative cosmetics. Preference is given to those cosmetic and dermatological products in the form of a cosmetic product which are used to convey a refreshing/cooling sensation to the human body, in particular the human skin.

The following examples illustrate the various aspects of the present invention. Unless indicated otherwise all parts, amounts and percentages are by weight.

### EXAMPLE 1

A mixture of 76.9 g racemic neomenthol, 14.8 g racemic menthol, 6.6 g racemic neoisomenthol and 1.7 g racemic isomenthol were stirred resulting in a liquid mixture (Mix A). Alternatively such a liquid mixture can be obtained via hydrogenation of thymol (see WO 2004/018398) and subsequent distillation.

25 g of l-menthyl l-lactate were warmed to 45-50° C. to liquefy the l-menthyl l-lactate and added with stirring to 75 g of Mix A.

The resulting clear solution was allowed to cool and then stored at −20° C. for 48 h. No precipitation or solidification was observed.

### EXAMPLE 2

A mixture of 67.8 g racemic neomenthol, 25.6 g racemic menthol, 5.6 g racemic neoisomenthol and 1 g racemic isomenthol were stirred resulting in a liquid mixture (Mix B). Alternatively such a liquid mixture can be obtained via hydrogenation of thymol (see WO 2004/018398) and subsequent distillation.

15 g of l-menthyl l-lactate were warmed to 45-50° C. to liquefy the l-menthyl l-lactate and added with stirring to 85 g of Mix B.

The resulting clear solution was allowed to cool and then stored at −20° C. for 48 h. No precipitation or solidification was observed.

### EXAMPLE 3

A mixture of 50 g racemic neomenthol and 50 g racemic menthol were stirred resulting in a liquid mixture (Mix C).

25 g of l-menthyl l-lactate were warmed to 45-50° C. to liquefy the l-menthyl l-lactate and added with stirring to 75 g of Mix C.

The resulting clear solution was allowed to cool and then stored at −20° C. for 48 h. No precipitation or solidification was observed.

### COMPARATIVE EXAMPLE

To illustrate the solidification/precipitation of l-menthyl l-lactate in racemic menthol alone, 25 g of l-menthyl l-lactate were liquefied by heating to 45-50° C. and mixed at 45-50° C. with 75 g racemic menthol. Upon cooling and storage at 0° C. for 8 h the mixture had completely solidified.

According to US 2004/0018954 a mixture consisting of 25 g l-menthyl l-lactate and 75 g l-menthol has a solidification point of 21.1° C.

### EXAMPLE 4

A sensory test was carried out with a group of 19 trained panelists in order to evaluate the performance of the coolant composition according to the present invention of Example 2 consisting of 15% l-menthyl l-lactate, 57.6% racemic neomenthol, 21.8% racemic menthol, 4.8% racemic neoisomenthol and 0.8% racemic isomenthol) for topical applications.

The cooling sensation of the coolant composition of Example 2 was tested in direct comparison to l-menthyl l-lactate. The mixture according to Example 2 and l-menthyl l-lactate were separately incorporated into samples of an alcohol-free face lotion at a level of 0.3%, based on the total weight of the face lotion.

Although neomenthol does not have a strong cooling sensation (vide supra) the panelists found a significantly higher overall cooling sensation and preferred the cooling sensation of the coolant composition of Example 2 according to the present invention.

The presented data are average values. The overall cooling sensation was rated on a scale of 1 to 5 (1=very weak; 5=very strong).

| | | 0.3% of the coolant |
|---|---|---|
| | 0.3% I-menthyl I- | composition according |
| | lactate | to Example 2 |
| Overall cooling sensation | 2.4 | 3.3 |
| (facial skin) | | |
| When does the cooling | after 1 minute | immediately |
| sensation become noticeable? | | |
| How long does the cooling | about 9 minutes | about 8 minutes |
| sensation last ? | | |
| Was the cooling sensation | yes: 73.7% | yes: 84.2% |
| pleasant ? | | |

### EXAMPLE 5

A sensory test was carried out with a group of 10 trained panelists in order to evaluate the performance of the liquid coolant compositions of Examples 1 and 2. Panelists were asked to assess the intensity of cooling sensation at certain time periods on a line scale of zero to one hundred. Panelists held toothpaste containing formulations of Example 1 (0.6% by weight based on total weight of the toothpaste) and Example 2 (0.6% by weight based on the total weight of the toothpaste) in their mouths for 90 seconds before spitting and rinsing. Panelists recorded the cooling sensation at rinse (time zero), 30 s, 60 s, 90 s, 2 min, 3 min, 4, min, 5 min, 10 min, 15 min, 20 min, 25 min and 30 min.

Toothpastes containing a coolant composition produced according to Example 1 or Example 2 both outperformed toothpastes containing only menthyl lactate or containing only racemic neomenthol and/or racemic menthol.

In the following examples reference is made to “Fresc 36-07” or “Fresc 36-37”, both being compositions according to the present invention.

“Fresc 36-07” is a mixture of 15% of l-menthyl l-lactate and 85% of a mixture of menthol isomers consisting of 67.6% racemic neomenthol, 25.6% racemic menthol, 5.5% racemic neoisomenthol, 0.7% racemic isomenthol, 0.4% racemic menthone and 0.2% racemic isomenthone.

“Fresc 36-37” is a mixture of 15% of l-menthyl l-lactate and 85% of a mixture of menthol isomers consisting of 78.0% racemic neomenthol, 13.7% racemic menthol, 5.7% racemic neoisomenthol, 1.0% racemic isomenthol, 1.1% racemic menthone and 0.5% racemic isomenthone.

### EXAMPLE 6

Sprayable Refreshing Body Lotion (O/W) with Fresc 36-07

| | Ingredients | INCI-Name | % (w/w) |
|---|---|---|---|
| A | Demineralised Water | Water (Aqua) | 72.20 |
| | Glycerin 99% | Glycerin | 4.00 |
| | 1,3-Butylene Glycol | Butylene Glycol | 5.00 |
| | D-Panthenol | Panthenol | 1.00 |
| | Allantoin | Allantoin | 0.10 |
| | Lara Care A-200 | Galactoarabinan | 0.25 |
| | Aloe Vera-Gel- | Water (Aqua), Aloe Barbadensis | 3.00 |
| | Concentrate 10/1 | Leaf Juice | |
| B | Baysilone Oil M 350 | Dimethicone | 1.00 |
| | Cetiol OE | Dicaprylyl Ether | 4.00 |
| | Cetiol SB 45 | Butyrospermum Parkii | 1.00 |
| | | (Shea Butter) | |
| | Tegosoft TN | C12-15 Alkyl Benzoate | 5.00 |
| | Copherol 1250 | Tocopheryl Acetate | 0.50 |
| | Alpha Bisabolol, | Bisabolol | 0.10 |
| | natural | | |
| | Fragrance | Fragrance | 0.30 |
| | Fresc 36-07 | | 1.00 |
| | Pemulen TR 2 | Acrylates/C10-30 Alkyl Acrylate | 0.25 |
| | | Crosspolymer | |
| | Dragocide Liquid | Phenoxyethanol, Methylparaben, | 0.70 |
| | | Ethyl-paraben, Butylparaben, | |
| | | Propylparaben, Isobutylparaben | |
| C | Sodium Hydroxide | Sodium Hydroxide | 0.60 |
| | 10% aq. solution | | |

Manufacturing Process

### Part A:

Dissolve all ingredients in the water.

### Part B:

Dissolve Cetiol SB 45 with heating to max. 35° C. in Tegosoft TN. Add the other ingredients of part B at ambient temperature. Disperse Pemulen evenly with high shear stirring. Add part B with stirring to part A and homogenize.

### Part C:

Add with stirring to part A/B and homogenize.

The pH value of the finished product should be approx. 6.0 and has to be checked.

### EXAMPLE 7

Face Cream (O/W) with Fresc 36-07

| | Ingredients | INCI-Name | %(w/w) |
|---|---|---|---|
| A | Dracorin GMS | Glyceryl Stearate | 2.00 |
| | Emulsiphos | Potassium Cetyl Phosphate, | 2.00 |
| | | Hydrogenated Palm Glycerides | |
| | PCL-Solid | Stearyl Heptanoate, Stearyl | 1.00 |
| | | Caprylate | |
| | Lanetta O | Cetearyl Alcohol | 2.00 |
| | Neutral Oil | Caprylic/Capric Triglycerides | 3.00 |
| | Tegosoft TN | C12-15 Alkyl Benzoate | 3.00 |
| B | Water | Water (Aqua) | 84.15 |
| | Carbopol ETD 2001 | Carbomer | 0.20 |
| | Keltrol T | Xanthan Gum | 0.20 |
| | Dragocide Liquid | Phenoxyethanol, Methylparaben, | 0.80 |
| | | Ethyl-paraben, Butylparaben, | |
| | | Propylparaben, Isobutylparaben | |
| | D-Panthenol 75 L | Panthenol | 0.50 |
| C | Sodium Hydroxide | Sodium Hydroxide | 0.45 |
| | 10% aq. solution | | |
| D | Fragrance | Fragrance | 0.30 |
| | Alpha Bisabolol, | Bisabolol | 0.10 |
| | natural Fresc 36-07 | | 0.30 |

Manufacturing Process

### Part A:

Heat to approx. 80° C.

### Part B and C:

Swell Carbopol and Keltrol in water. Heat to approx. 80° C. Add part A to part B using an Ultra-Turrax and emulsify, add part C while emulsifying. Allow to cool while stirring.

### Part D:

Mix the ingredients and add at approx. 30° C. to the emulsion and homogenise.

The pH value of the finished product should be approx. 5.7 and has to be checked.

### EXAMPLE 8

Transparent Hair Shampoo with Fresc 36-37

| | Ingredients | INCI-Name | % (w/w) |
|---|---|---|---|
| A | Genapol LRO liquid | Sodium Laureth Sulfate | 40.00 |
| | Dehyton K | Cocamidopropyl Betaine | 7.00 |
| | Akyposoft 100 BVC | Sodium Laureth-11 Carboxylate, | 8.50 |
| | | Laureth-10 | |
| | Arlypon F | Laureth-2 | 2.50 |
| | Dragocide Liquid | Phenoxyethanol, Methylparaben, | 0.70 |
| | | Ethyl-paraben, Butylparaben, | |
| | | Propylparaben, Isobutylparaben | |
| B | Fragrance | Fragrance | 0.50 |
| | Fresc 36-37 | | 0.80 |
| | Arlatone G | PEG-25 Hydrogenated Castor Oil | 0.50 |
| C | Demineralised | Water (Aqua) | 37.10 |
| | Water | | |
| | Polymer JR 400 | Polyquaternium-10 | 0.30 |
| | Sodium Hydroxide | Sodium Hydroxide | 0.10 |
| | 10% aq. Solution | | |
| | Extrapone Seaweed | Water (Aqua), Propylene Glycol, | 1.00 |
| | | Potassium Iodide, Fucus | |
| | | Vesiculosus Extract | |
| | Extrapone Ginkgo | Propylene Glycol, Water (Aqua), | 1.00 |
| | Biloba | Ginkgo Biloba Leaf Extract, | |
| | | Glucose, Lactic Acid | |

Manufacturing Process

### Part A:

Mix all ingredients.

### Part B:

Mix Fresc 36-07 with Arlatone G and the fragrance. Then add to part A.

### Part C:

Dissolve Polymer JR 400 in the water. Add the other ingredients and add part C to part A/B. Stir until homogeneous.

The pH value of the finished product should be approx. 6.0 and has to be checked.

### EXAMPLE 9

Body Wash with Fresc 36-07

| | Ingredients | INCI-Name | % (w/w) |
|---|---|---|---|
| A | Dehyton K | Cocamidopropyl Betaine | 10.00 |
| | Plantacare 1200 | Lauryl Glucoside | 5.00 |
| | UP | | |
| | Cutina AGS | Glycol Distearate | 2.00 |
| B | Texapon NSO | Sodium Laureth Sulfate | 30.00 |
| | Dragocide Liquid | Phenoxyethanol, Methylparaben, | 0.80 |
| | | Ethyl-paraben, Butylparaben, | |
| | | Propylparaben, Isobutylparaben | |
| | Fresc 36-07 | | 0.40 |
| | Merquat 550 | Polyquaternium-7 | 1.00 |
| | Extrapone | Glycerin, Water (Aqua), Prunus | 1.00 |
| | Almond Milk | Amygdalus Dulcis (Sweet Almond) | |
| | | Seed Extract, Propylene Glycol, | |
| | | PEG-40 Hydrogenated Castor Oil, | |
| | | Trideceth-10, Nonfat Dry Milk (Sine | |
| | | Apide Lac), Prunus Amygdalus | |
| | | Dulcis (Sweet Almond) Oil | |
| | Citric Acid, 10% | Citric Acid | 1.10 |
| | aq. solution | | |
| | Water | Water (Aqua) | 47.30 |
| | Sodium Chloride | Sodium Chloride | 0.90 |
| | Fragrance | Fragrance | 0.50 |

Manufacturing Process

Dissolve phase A by warming up slightly. Add ingredients of phase B in listed order and dissolve.

The pH value of the finished product should be approx. 5.5 and has to be checked.

### EXAMPLE 10

Hair Tonic with Fresc 36-37

| | Ingredients | INCI-Name | % (w/w) |
|---|---|---|---|
| A | Ethyl alcohol (96 | SD-Alcohol 39-C | 50.00 |
| | Vol. %) denatured | | |
| | Diisopropyl adipate | Diisopropyl Adipate | 1.00 |
| | Salicylic Acid | Salicylic Acid | 0.20 |
| | Cremophor RH 455 | PEG-40 Hydrogenated Castor Oil, | 0.30 |
| | | Propylene Glycol | |
| | Fragrance | Fragrance | 0.30 |
| | Crinipan ® AD | Climbazole | 0.30 |
| | Fresc 36-37 | | 0.60 |
| B | Demineralised | Water (Aqua) | 41.50 |
| | Water | | |
| | D-Panthenol | Panthenol | 0.50 |
| | Dehyquart A | Cetrimonium Chloride | 0.20 |
| | Allantoin | Allantoin | 0.10 |
| | Glycerin 85/86% | Glycerin | 2.00 |
| | Extrapone Birch | Water (Aqua), Propylene Glycol, | 3.00 |
| | | Betula Alba Leaf Extract, | |
| | | Betula Alba Juice, SD Alcohol | |
| | | 39-C (Alcohol Denat.) | |

Manufacturing Process

### Part A:

Dissolve Crinipan® AD and Fresc 36-07 in the other ingredients of part A.

### Part B:

Mix all ingredients and add to part A.

The pH value of the finished product should be approx. 6.5 and has to be checked.

### EXAMPLE 11

Sunscreen Lotion with Fresc 36-07

| | Ingredients | INCI-Name | % (w/w) |
|---|---|---|---|
| A | Emulsiphos | Potassium Cetyl Phosphate, | 1.50 |
| | | Hydrogenated palm Glycerides | |
| | Tegosoft TN | C12-15 Alkyl Benzoate | 4.00 |
| | Copherol 1250 | Tocopheryl Acetate | 0.50 |
| | Lanette O | Cetearyl Alcohol | 1.00 |
| | Dow Corning 246 | Cyclohexasiloxane, | 2.00 |
| | Fluid | Cyclopentasiloxane | |
| | Neo Heliopan ® 357 | Butyl | 1.00 |
| | | Methoxydibenzoylmethane | |
| | Neo Heliopan ® AV | Ethylhexyl Methoxycinnamate | 3.00 |
| | Neo Heliopan ® OS | Ethylhexyl Salicylate | 5.00 |
| | Edeta BD | Disodim EDTA | 0.10 |
| | Keltrol T | Xanthan Gum | 0.20 |
| | Carbopol ETD 2001 | Carbomer | 0.20 |
| B | Demineralised Water | Water (Aqua) | 63.48 |
| | Glycerin 99.5 P. | Glycerin | 4.70 |
| | Dragocide Liquid | Phenoxyethanol, Methylparaben, | 0.70 |
| | | Ethyl-paraben, Butylparaben, | |
| | | Propylparaben, Isobutylparaben | |
| | Neo Heliopan ® AP, | Disodium Phenyl | 4.55 |
| | used as a 22% solution | Dibenzimidazole Tetrasulfonate | |
| | neutralised with | | |
| | triethanolamine | | |
| | Neo Heliopan ® | Phenylbenzimidazole | 6.67 |
| | Hydro, used as a 30% | Sulfonic Acid | |
| | solution neutralised | | |
| | with triethanolamine | | |
| C | Triethanolamine, 99% | Triethanolamine | 0.50 |
| D | Fragrance | Fragrance | 0.40 |
| | Dragosantol | Bisabolol | 0.10 |
| | Fresc 36-07 | | 0.40 |

Manufacturing Process

### Part A:

Heat up to 85° C. (without Keltrol and carbopol) until all ingredients completely dissolved. Disperse Keltrol and Carbopol in part A.

### Part B:

Heat part B to 85° C. Add part B to part A while stirring.

### Part C:

Add directly. Homogenise the product for 5 minutes. Cool down to ambient temperature.

### Part D:

At ambient temperature add part D and homogenise.

The pH value of the finished product should be approx. 7.2 to 7.5 and has to be checked.

### EXAMPLE 12

Shower Gel with Fresc 36-07

| | Ingredients | INCI-Name | % (w/w) |
|---|---|---|---|
| A | Genapol LRO liquid | Sodium Laureth Sulfate | 40.00 |
| | Dehyton K | Cocamidopropyl Betaine | 7.00 |
| | Akyposoft 100 BVC | Sodium Laureth-11 Carboxylate, | 8.50 |
| | | Laureth-10 | |
| | Arlypon F | Laureth-2 | 2.50 |
| | Dragocide Liquid | Phenoxyethanol, Methylparaben, | 0.50 |
| | | Ethylparaben, Butylparaben, | |
| | | Propylparaben, Isobutylparaben | |
| B | Fragrance | Fragrance | 1.00 |
| | Fresc 36-07 | | 1.20 |
| | Arlatone G | PEG-25 Hydrogenated Castor Oil | 0.50 |
| C | Demineralised | Water (Aqua) | 36.40 |
| | Water | | |
| | Polymer JR 400 | Polyquaternium-10 | 0.30 |
| | Sodium Hydroxide | Sodium Hydroxide | 0.10 |
| | 10% aq. Solution | | |
| | Extrapone Seaweed | Water (Aqua), Propylene Glycol, | 1.00 |
| | | Potassium Iodide, Fucus | |
| | | Vesiculosus Extract | |
| | Extrapone Ginkgo | Propylene Glycol, Water (Aqua), | 1.00 |
| | Biloba | Ginkgo Biloba Leaf Extract, | |
| | | Glucose, Lactic Acid | |

Manufacturing Process

### Part A:

Mix all ingredients.

### Part B:

Mix the ingredients. The add part B to part A.

### Part C:

Dissolve Polymer JR 400 under stirring in the water. Add the other ingredients of part C and add to part A/B. Stir until homogeneous.

The pH value of the finished product should be approx. 6 and has to be checked.

### EXAMPLE 13

Shaving Foam with Fresc 36-37

| | Ingredients | INCI-Name | % (w/w) |
|---|---|---|---|
| A | Demineralised Water | Water (Aqua) | 81.80 |
| | Karion F | Sorbitol | 3.00 |
| | Myvatex Texture Lite | Glycerol Monstearate, Sodium | 4.50 |
| | | Stearoyl Lactylate | |
| | Solbrol M | Methylparaben | 0.15 |
| B | Myverol 18.92 | Sunflower Seed Oil Glyceride | 2.00 |
| | Lanette 16 | Cetyl Alcohol | 1.50 |
| | Glycerin 99% | Glycerin | 3.50 |
| | Solbrol P | Propylparaben | 0.05 |
| C | Plantacare 1200 UPNP | Lauryl Glycoside | 1.50 |
| D | Frangrance | Fragrance | 0.50 |
| | Fresc 36-37 | | 1.50 |

Manufacturing Process

### Part A:

Dissolve Solbrol M in Sorbitol A, add the water. With mixing slowly add Myvatex Texture Lite until well dispersed. Heat up to 40° C. (do not exceed 45° C.).

### Part B:

Combine and heat up to 60° C. Mix until uniform. Cool down to 50° C. and add part A to part B while stirring.

### Part C:

Add part C to part A/B and continue mixing for approx. 20 minutes.

### Part D:

Add to part A/B/C. Fill into aerosol containers.

### Filling Instructions:

95.30% filling composition

4.70% Propan/butan 2.7 bar

Remark on the can: Shake before use!

The pH value of the finished product should be approx. 7.0 and has to be checked.

### EXAMPLE 14

Mouthwash with Fresc 36-07

| | Ingredients | INCI-Name | % (w/w) |
|---|---|---|---|
| A | Aroma Oil | Aroma | 0.30 |
| | Fresc 36-07 | | 0.20 |
| | Cremophor RH 40 | PEG-40 Hydrogenated | 2.00 |
| | | Castor Oil | |
| | Cetylpyridinium Chloride | Cetylpyridinium Cloride | 0.20 |
| | Monohydrate | | |
| | Water | Water (Aqua) | qs |
| | 1,2-Propylene Glycol | Propylene Glycol | 1.00 |
| | Glycerin 99.5 P. | Glycerin | 2.00 |
| | Karion F | Sorbitol | 10.00 |
| | Colour solution | Colour | qs |
| | PVP-K | PVP | 0.10 |

Manufacturing Process

Add ingredients as listed and dissolve. The solution should be cooled down to +5° C. for 24 hours, afterwards it should be filtered.

### EXAMPLE 15

Deodorant/Antiperspirant Roll-on with Fresc 36-07

| | Ingredients | INCI-Name | % (w/w) |
|---|---|---|---|
| A | Lanette 16 | Cetyl Alcohol | 2.50 |
| | Eumulgin B2 | Ceteareth-20 | 2.00 |
| | Dracorin GMS | Glyceryl Stearate | 2.00 |
| | PCL Liquid 100 | Cetearyl Ethylhexanoate | 1.00 |
| B | Deolite | Penthylene Glycol, Dimethyl | 0.50 |
| | | Phenylpropanol | |
| | Water | Water (Aqua) | 64.40 |
| | Locron L | Aluminium Chlorohydrate | 25.00 |
| | Aloe Vera-Gel- | Water (Aqua), Aloe | 1.00 |
| | Concentrate 10/1 | Barbadensis Leaf Juice | |
| C | Fragrance | Fragrance | 1.00 |
| | Fresc 36-07 | | 0.60 |

Manufacturing Process

Heat Phases A and B separately to approx. 80° C. Then add phase B to phase A and emulsify (Ultra-Turrax). Allow the emulsion to cool while stirring using a vane stirrer. Reduce stirring speed while the base is cooling. Add phase C at approx. 30° C.

1. A composition comprising
(a) menthyl lactate
(b) neomenthol, and
(c) menthol,
and optionally
(d) neoisomenthol and/or (e) isomenthol,
wherein said composition has a solidification point below +5° C.

2. A composition according to claim 1, wherein the weight ratio of component (a) to the sum of components (b) and (c) is in the range of from 6:1 to 1:15, and the weight ratio of components b) to c) is in the range of from 20:1 to 1:10.

3. A composition according to claim 1, wherein the added amount of components (a), (b) and (c) is in the range of from 80 to 100% by weight, based on the total weight of the composition.

4. A composition according to claim 1, comprising
10-40% of menthyl lactate,
25-75% of neomenthol,
5-30% of menthol,
0-10% of neoisomenthol, and
0-10% of isomenthol, by weight, based on the total weight of the composition.

5. A composition according to claim 1, wherein component (a) comprises l-menthyl l-lactate.

6. A product comprising (i) a product base and (ii) an amount of a composition according to claim 1 sufficient to provide a physiological cooling effect.

7. A method of preparing a product, comprising:
storing a composition according to claim 1 at a temperature (i) below +5° C. but (ii) above the solidification point of said composition,
mixing the composition with components of a product base to form said product,
wherein said composition is not solidified between storing and mixing.
